(19) United States
(12) Patent Application Publication
(10) Pub. No.: US 2022/0266245 A1
(43) Pub. Date: Aug. 25, 2022

(54) A MAGNETIC DIGITAL MICROFLUIDIC SYSTEM AND METHOD OF PERFORMING AN ASSAY

(71) Applicants: Tan Tock Seng Hospital Pte Ltd, Singapore (SG); Nanyang Technological University, Singapore (SG)

(72) Inventors: Yi ZHANG, Singapore (SG); Pojchanun KANITTHAMNIYOM, Singapore (SG); Shawn VASOO, Singapore (SG)

(21) Appl. No.: 17/626,080

(22) PCT Filed: Sep. 1, 2020
(86) PCT No.: PCT/SG2020/050510

§ 371 Date: Jan. 10, 2022

(30) Foreign Application Priority Data
Sep. 2, 2019 (SG) 10201908072Y

(51) Int. Cl. B01L 3/00

(52) U.S. Cl. CPC B01L 3/50273; B01L 3/502792; B01L 3/502761; B01L 2200/027; B01L 2400/043

(57) ABSTRACT

There is provided a magnetic digital microfluidic system for performing an assay, the system comprising, abase member comprising at least one magnet disposed thereon; the magnet being configured to immobilise a droplet of reaction mixture doped with magnetic particles; and a droplet manipulator configured to be moveably mountable on the base member, said droplet manipulator comprising at least one test unit, each test unit comprising at least one mixing element for mixing the droplet of reaction mixture; wherein the mixing element is arranged to induce mixing as the droplet manipulator is moved in relation to the base member along an alignment path. There is also provided a method of performing an assay with the magnetic digital microfluidic system as disclosed herein.

### TECHNICAL FIELD

The present disclosure relates broadly to a magnetic digital microfluidic system and method of performing an assay.

### BACKGROUND

In general, point-of care diagnostics should be based on systems/platforms that are relatively simple and robust enough to be operated with minimal or no training. In addition, the systems for point-of care diagnostics should be capable of performing on-site testing where there may be inadequate infrastructure such as trained personnel, power source, and/or equipment. The systems for point-of care diagnostics should also be relatively low cost in terms of material and production, so that mass production and/or disposable usage of components in the systems are feasible.

Conventional microfluidic systems often require complicated fluidic pumping and valving mechanisms for fluidic control. Precise and controlled microfluidic pumping of fluids are typically required in order to reliably perform an assay using conventional microfluidic systems. However, fluid manipulation in the microfluidic system using external pumps e.g. syringe pump and peristaltic pump may be difficult for untrained personnel to handle. In addition, the requirement of external power sources and/or peripheral control systems may result in conventional microfluidic systems becoming bulky, complex, and/or costly to use. Hence, conventional microfluidic systems may not be well suited for point-of-care diagnostics.

In addition, some detection assays such as the Carba NP assay are tedious to perform and typically only analyze one clinical isolate at a time, which can be time-consuming in diagnostic settings where a large number of clinical isolates need to be tested. It is therefore important that detection assays for point-of-care diagnostics are capable of analyzing multiple samples concurrently and generate results within a relatively short period of time.

Thus, there is a need for a system and a method of performing an assay that seek to address or at least ameliorate one or more of the above problems.

### SUMMARY

In one aspect, there is provided a magnetic digital microfluidic system for performing an assay, the system comprising, a base member comprising at least one magnet disposed thereon; the magnet being configured to immobilise a droplet of reaction mixture doped with magnetic particles; and a droplet manipulator configured to be moveably mountable on the base member, said droplet manipulator comprising at least one test unit, each test unit comprising at least one mixing element for mixing the droplet of reaction mixture; wherein the mixing element is arranged to induce mixing as the droplet manipulator is moved in relation to the base member along an alignment path.

In one embodiment, the system further comprises a surface for allowing the droplet of reaction mixture to be disposed thereon.

In one embodiment of the system as disclosed herein, the surface is a detachable surface and the droplet manipulator is configured to detachably couple with the detachable surface.

In one embodiment of the system as disclosed herein, the test unit comprises, a first access port for allowing delivery of a sample, magnetic particles, and/or one or more reaction reagents to said surface.

In one embodiment of the system as disclosed herein, the mixing element comprises an array of pillars being arranged to interact with the droplet of reaction mixture to induce mixing.

In one embodiment of the system as disclosed herein, the test unit further comprises a second access port for allowing delivery of a detection reagent to said surface.

In one embodiment of the system as disclosed herein, the test unit further comprises a droplet holder configured to engage and hold the droplet of reaction mixture to facilitate observation.

In one embodiment of the system as disclosed herein, the droplet holder comprises a hydrophilic contact surface for engaging and holding the droplet of reaction mixture.

In one embodiment of the system as disclosed herein, the droplet holder is further configured to facilitate removal of the magnetic particles from the droplet of reaction mixture via movement of the droplet manipulator along the alignment path.

In one embodiment of the system as disclosed herein, the test unit further comprises an observation window configured to facilitate observation of the droplet of reaction mixture.

In one embodiment of the system as disclosed herein, the droplet manipulator comprises a plurality of test units for performing the assay on a plurality of samples simultaneously, each test unit being configured to cooperate with a respective magnet disposed on the base member.

In one embodiment, the system further comprises a guide member configured to be detachably couplable to the droplet manipulator, said guide member comprising at least one guide hole configured to be alignable with the first access port, wherein the guide hole is dimensioned such that a droplet dispenser coupled thereto does not contact the surface where the droplet is to be disposed.

In one embodiment of the system as disclosed herein, said guide member comprises at least one guide hole configured to be alignable with the second access port, wherein the guide hole is dimensioned such that a droplet dispenser coupled thereto does not contact the surface where the droplet is to be disposed.

In one aspect, there is provided a method of performing an assay with the magnetic digital microfluidic system as disclosed herein, the method comprising, moving the droplet manipulator in relation to the base member along an alignment path to induce mixing of a droplet of reaction mixture immobilised by the magnet disposed on the base member, wherein the reaction mixture is doped with magnetic particles; and observing changes to the reaction mixture.

In one embodiment, the method further comprises, prior to the moving step, disposing a droplet of reaction mixture on a surface that is coupled to the droplet manipulator via a first access port of the test unit.

In one embodiment, the method further comprises, subsequent to said moving step, disposing a droplet of detection reagent on said surface via a second access port of the test unit; further moving the droplet manipulator in relation to the base member along an alignment path to merge the droplet of reaction mixture with the droplet of detection reagent; and optionally further moving the droplet manipulator in relation to the base member along an alignment path to further mix the merged droplet.

In one embodiment, the method further comprises, prior to the each of said disposing step(s), detachably coupling a guide member to the droplet manipulator; and aligning a guide hole of the guide member to the access port through which the respective disposing step is to be carried out, wherein the guide hole is dimensioned such that a droplet dispenser does not contact the surface of the droplet manipulator when disposing the reaction mixture or detection agent thereon.

In one embodiment, the method further comprises, detaching the guide member from the system prior to each of said moving step(s).

In one embodiment, the method further comprises, moving the droplet manipulator in relation to the base member along an alignment path to engage and hold the droplet of reaction mixture with a droplet holder; moving the droplet manipulator in relation to the base member along an alignment path to remove the magnetic particles from the droplet of reaction mixture; and observing via an observation window in the test unit, the droplet of reaction mixture that is engaged and held by the droplet manipulator.

In one embodiment, the method further comprises performing the assay on a plurality of samples simultaneously on a plurality of test units in the droplet manipulator, wherein each test unit is being configured to cooperate with a respective magnet disposed on the base member.

### DEFINITIONS

The term “sample” as used herein refers to any matter or composition that might contain a target of interest to be analyzed. The term “sample” includes, but is not limited to, biological samples obtained from subjects (including humans and animals), samples obtained from the environment for example soil samples, water samples, samples obtained from various surfaces at a location e.g. hospital, or food samples.

The term “antimicrobial agent” as used herein refers to an agent which kills or inhibits the growth of a microorganism, including for example bacteria, yeast, fungi, viruses, parasites, etc. An antimicrobial agent which inhibits growth of a microorganism or population of microorganism is said to be microbiostatic (e.g. bacteriostatic in the case of an antibacterial agent which inhibits the growth of bacteria). An antimicrobial agent which kills a microorganism or population of microorganism is said to be microbiocidal (e.g. bacteriocidal in the case of an antibacterial agent which kills bacteria).

The term “magnetic” as used herein refers to magnetic properties of a material. The term “magnetic particles” as used herein refers to particles that are made from materials that possess magnetic properties. Magnetic particles are capable of interacting with a magnetic field, generating either an attractive force or a repulsive force. If a magnetic field is applied to a magnetic particle, the particle becomes magnetized. The magnetized particle may be classified into ferromagnetic, paramagnetic or superparamagnetic, depending on the type of materials and magnetization resulting from the application of a magnetic field. A ferromagnetic material is a substance which is strongly magnetized in the same direction as a magnetic field when a strong magnetic field is externally applied and remains magnetized even after the external magnetic field is removed. Examples of the ferromagnetic material include iron, cobalt, nickel, alloys thereof, and the like. A paramagnetic material is a substance which is weakly magnetized in the same direction as an external magnetic field upon application of the magnetic field and has no remaining magnetism after the external magnetic field is removed. Examples of the paramagnetic material include metals such as aluminum, tin, platinum and iridium. Magnetic particles may have average diameters in the range of nanometers, micrometers, or millimeters. Magnetic particles may include magnetic microbeads and/or nanoparticles.

The term “droplet” as used herein means a discrete volume of liquid, i.e. a discrete droplet of liquid. The volume of liquid may be in the range of nano-liters, microliters, or milliliters. Droplets may, for example, be aqueous or non-aqueous or may be mixtures or emulsions including aqueous and non-aqueous components. Droplets may take on various shapes which may include, but are not limited to, disc shaped, slug shaped, truncated sphere, ellipsoid, spherical, partially compressed sphere, hemispherical, and ovoid.

The term “immobilise” as used herein means to substantially restrain an object in a specific position, thereby substantially preventing the object from moving away from the specific position. The specific position may be one that is in relation to a specific point of reference. For example, in various embodiments disclosed herein, the specific point of reference may be a position of a magnet. Therefore, an immobilised object may not substantially change its position relative to its specific point of reference but may change its position relative to other reference points.

The term “substrate” as used herein is to be interpreted broadly to refer to any supporting structure.

The term “substantially transparent to light” when used herein to describe an object is to be interpreted broadly to mean that 50% or more of the incident light normal to surface of the object can be transmitted through the object. In some examples, the object that is substantially transparent to light allow 60% or more, 65% or more, 70% or more, 80% or more, 85% or more, 90% or more or 95% or more of the incident light normal to surface of the object to be transmitted. In one example, the object that is substantially transparent to light allow above 70% of the incident light normal to surface of the object to be transmitted.

The term “translucent” when used herein to describe a material is to be interpreted to mean that the material transmits and diffuses light rays so that objects beyond the material cannot be seen clearly through the material.

The term “micro” as used herein is to be interpreted broadly to include dimensions from about 1 micron to about 1000 microns.

The term “nano” as used herein is to be interpreted broadly to include dimensions less than about 1000 nm.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Non-limiting embodiments of a system and method of performing an assay are disclosed hereinafter.

In various embodiments, there is provided a system for performing an assay, the system comprising, a base member comprising at least one magnet disposed thereon; the magnet being configured to immobilise a droplet of reaction mixture doped with magnetic particles; and a droplet manipulator configured to be moveably mountable on the base member, said droplet manipulator comprising at least one test unit, each test unit comprising at least one mixing element for mixing the droplet of reaction mixture; wherein the mixing element is arranged to induce mixing as the droplet manipulator is moved in relation to the base member along an alignment path.

In various embodiments, the droplet manipulator is configured to be slidably mountable on the base member and the mixing element is arranged to induce mixing as the droplet manipulator is slided back and forth in relation to the base member along the alignment path. In various embodiments, the alignment path may be a linear or non-linear path e.g. circular path or part thereof.

In various embodiments, the base member functions to provide a surface for allowing the droplet manipulator to be moveably or slidably mounted thereon. The base member may comprise a substantially flat surface for allowing the droplet manipulator to move or slide thereon along the alignment path. The base member may further comprise protrusions disposed on the substantially flat surface. For example, the protrusions may define a first side wall and a second side wall disposed at opposite ends of the substantially flat surface. The first and second side walls may function to define the range of movement of the droplet manipulator along the alignment path on the base member. The base member may comprise a third side wall substantially perpendicular to, and connecting the first and second side walls. The third side wall may function to facilitate alignment of the droplet manipulator on the base member and movement of the droplet manipulator along the alignment path. In various embodiments, it will be appreciated that the base member is in a stationary position as the droplet manipulator is moved along the alignment path.

In various embodiments, the base member comprises at least one magnet disposed thereon. The base member may comprise a plurality of magnets disposed thereon. The plurality of magnets disposed on the base member may be arranged in an array (i.e. in an orderly manner) such that each magnet is positioned to be spaced apart from adjacent neighbouring magnets at regular intervals. In various embodiments, the magnet(s) disposed on the base member may be fixed in position on the base member, i.e. not moveable with respect to the base member. In one example, the magnet may be positioned on top of the substantially flat surface of the base member such that the magnet is protruding from (i.e. forms a protrusion on) the substantially flat surface. In another example, the magnet may be embedded in the substantially flat surface of the base member. A hole/space may be provided in the base member for accommodating the magnet such that the magnet does not protrude from the substantially flat surface. In various embodiments, this allows the droplet manipulator to move or slide smoothly along the substantially flat surface of the base member. The magnet that is embedded in the substantially flat surface of the base member may be exposed (i.e. uncovered), partially exposed, or completely covered by material of the base member.

In various embodiments, the magnet functions to manipulate e.g. immobilise a droplet which is doped with magnetic particles by applying a magnetic field which exerts a magnetic force on the magnetic particles. The magnet may be of different shapes and sizes. For example, the magnet may be a rounded bar magnet. In various embodiments, the magnet may be disposed on the base member at a fixed position and applies a magnetic field in a region around the fixed position. When the droplet is positioned or brought to a position within the magnetic field, the magnetic particles within the droplet are attracted to the magnet. In various embodiments, the magnetic forces acting on the magnetic particles in the droplet causes the magnetic particles (and therefore, the droplet) to be immobilised in the position within the magnetic field. In various embodiments, the magnetic forces restrain the droplet doped with magnetic particles from moving away from the magnetic field applied by the magnet. In various embodiments, the droplet doped with magnetic particles may not substantially displace from the immobilised position during performance of the assay. That is, the droplet doped with magnetic particles may be immobilised in the fixed position relative to the magnet until the assay is completed. Once the assay is completed, the droplet may be displaced from the fixed position, e.g. by using a droplet holder to remove the droplet from the fixed position, for observation of the assay result. In various embodiments, the magnet may be oriented such that the poles (north and south poles) of the magnet are substantially perpendicular to the substantially flat surface of the base member. In this configuration, when the droplet doped with magnetic particles is positioned or brought to a position within the magnetic field, the droplet may be immobilised at a position in space above the magnet.

In various embodiments, the magnet may be a permanent magnet or an electromagnet. For example, the magnet may be a permanent magnet e.g. rounded bar permanent magnet which can be embedded in the hole/space provided in the base member. For example, the magnet may be an electromagnet comprising coils embedded within the base member and connected to an external power supply. For the case where the magnet is a permanent magnet, the system may be capable of performing the assay without an external power source.

In various embodiments, the magnetic particles are made from materials which are ferromagnetic or paramagnetic. Ferromagnetic materials include, but are not limited to, Cobalt (Co), Nickel (Ni), magnetite (γ-Fe₂O₃) or any alloy thereof. Paramagnetic materials include metals such as aluminum, tin, platinum and iridium. The magnetic particles may have an average diameter ranging from about 0.05 μm to about 50 μm, from about 0.1 μm to about 45 μm, from about 0.2 μm to about 40 μm, from about 0.5 μm to about 35 μm, from about 1 μm to about 30 μm, from about 2 μm to about 25 μm, from about 5 μm to about 20 μm, or from about 10 μm to about 15 μm.

In various embodiments, the droplet manipulator comprises at least one test unit for performing the assay on the sample. In various embodiments, the test unit is positioned on the droplet manipulator such that the test unit is capable of cooperating with the magnet disposed on the base member when the droplet manipulator is slidably mounted on the base member. The droplet manipulator may comprise a plurality of test units for performing the assay on a plurality of samples. The plurality of test units may be arranged on the droplet manipulator such that each test unit is capable of cooperating with a respective magnet from the plurality of magnets disposed on the base member, when the droplet manipulator is moveable or slidably mounted on the base member. This may advantageously allow the assay to be performed simultaneously or concurrently on multiple samples.

In various embodiments, the droplet manipulator comprises at least two test units, at least four test units, at least six test units, at least eight test units, at least ten test units, at least twelve test units, at least fourteen test units, at least sixteen test units, at least eighteen test units, at least twenty test units, at least twenty two test units, or at least twenty four test units. The plurality of test units may be positioned on the droplet manipulator to be compatible for use with dispensing devices known in the art, such as multichannel pipettes e.g. 6-, 8-, 12- or 16-channel pipettes. The plurality of test units may be arranged to form at least one column on the droplet manipulator. The plurality of test units in the same column may be concurrently loaded using the dispensing device. The plurality of test units may be arranged to form two columns on the droplet manipulator, a first column may be used for performing an assay on a test group and a second column may be used for performing the assay on a control group. Such an arrangement may facilitate easy observation of assay results.

In various embodiments, the test unit comprises a mixing element which functions to induce mixing in a reaction mixture comprised in a droplet doped with magnetic particles. In various embodiments, the mixing element is arranged to induce mixing as the droplet manipulator moves along the alignment path in relation to the base member. The mixing element may be arranged to move in tandem with the droplet manipulator along the alignment path. In various embodiments, as the mixing element moves in tandem with the droplet manipulator along the alignment path in a first direction, the mixing element interacts with the droplet of reaction mixture which is immobilised in a fixed position in space relative to the magnet disposed on the base member. The mixing element may be further arranged to move in tandem with the droplet manipulator along the alignment path in a second opposite direction to provide further interaction of the mixing element with the droplet of reaction mixture. The interaction of the mixing element with the droplet of reaction mixture may involve the mixing element stretching and retracting the droplet of reaction mixture, thereby agitating and inducing mixing of the contents (e.g. sample, reaction reagents and detector reagents) of the reaction mixture within the droplet. In various embodiments, repeated back and forth (or to and fro) movement or sliding of the droplet manipulator in the first direction and second direction along the alignment path agitates and induces mixing of the contents of the reaction mixture within the droplet of reaction mixture.

The mixing element may be chemically modified to further facilitate manipulation and interaction with the droplet of reaction mixture. For example, a surface of the mixing element may be chemically treated by coating with one or more fluoropolymers e.g. an amorphous fluoropolymer, and/or one or more fluorosilanes.

In various embodiments, the mixing element comprises at least one pillar/protruding element extending from a surface of the droplet manipulator. The mixing element may comprise a plurality or an array of pillars/protruding elements extending from a surface of the droplet manipulator. For example, the pillar(s)/protruding element(s) may extend from an undersurface of the droplet manipulator such that the pillar(s)/protruding element(s) forms an overhanging structure above the droplet. The plurality of pillars may be arranged in a linear manner (e.g. side by side along an imaginary line) or in a non-linear manner (e.g. in a staggered formation) or in an arrayed manner (e.g. X rows of Y protruding elements). In various embodiments, as the array of pillars passes through the droplet of reaction mixture, the pillars stretch and retract the droplet repetitively, thereby inducing mixing inside the droplet. The dimensions of the pillar may be in the order of micrometers or millimeters. The mixing element may have different shapes which include, but are not limited to, a cylindrical shape, conical shape, tetrahedral shape, and the like.

In various embodiments, the system or droplet manipulator further comprises a surface/substrate for allowing the droplet of reaction mixture to be disposed thereon. The surface may be an integrated surface that is part of the droplet manipulator or a removable/detachable surface that is configured to engage e.g. detachably couple with the droplet manipulator. In various embodiments, the surface for disposing the droplet of reaction mixture is a substantially flat surface which is arranged to be parallel to the ground when in use. In various embodiments, the surface for disposing the droplet of reaction mixture is capable of accommodating a plurality of droplets in a plurality of test units. The surface for for disposing the droplet of reaction mixture may be a substrate with relatively low surface tension and adhesion, allowing droplets to move along the surface with minimum friction. The surface for disposing the droplet of reaction mixture may be, or has been, treated e.g. chemically treated to reduce surface energy. The treated surface may have a relatively low surface energy, and may be hydrophobic and/or oleophobic, providing a “slippery” substrate for droplet movement. For example, the surface for disposing the droplet of reaction mixture may be coated with one or more fluoropolymers e.g. amorphous fluoropolymer and/or one or more fluorosilanes. The relatively low surface energy of the surface may promote droplet formation and may facilitate movement (e.g. passive movement) of the droplet of reaction mixture along the surface.

In various embodiments, the surface for disposing the droplet of reaction mixture is arranged to move in tandem with the droplet manipulator along the alignment path. When the surface for disposing the droplet of reaction mixture is moving along the alignment path, the magnet disposed on the base member is capable of maintaining the droplet of reaction mixture in the immobilised position within the magnetic field of the magnet. In various embodiments, this has an effect of the immobilised droplet of reaction mixture moving passively relative to the surface for disposing the droplet of reaction mixture. For example, when the surface for disposing the droplet of reaction mixture is moving in the first direction along the alignment path, this creates an effect of the immobilised droplet of reaction mixture “moving” along the surface in the second opposite direction along the alignment path. For example, when the surface for disposing the droplet of reaction mixture is moving in the second direction along the alignment path, this creates an effect of the immobilised droplet of reaction mixture “moving” along the surface in the first opposite direction along the alignment path.

In one embodiment, the surface for disposing the droplet of reaction mixture is an integrated component of the droplet manipulator. That is, the surface for disposing the droplet of reaction mixture may be formed together with the droplet manipulator as a single component. In another embodiment, the surface for disposing the droplet of reaction mixture is a detachable component. The detachable component may be in the form of a sheet member. The sheet member may include any suitable surface for allowing the droplet of reaction mixture to be disposed thereon and includes but is not limited to a slide e.g. glass slide or coverslip e.g. glass coverslip. The droplet manipulator may comprise slots or receptacles for receiving the sheet member and holding the sheet member in position, such that the sheet member is coupled to the droplet manipulator and arranged to move in tandem with the droplet manipulator along the alignment path.

In various embodiments, the test unit of the droplet manipulator further comprises a first access port configured to facilitate/allow delivery of a sample, magnetic particles, and/or one or more reaction reagents. The first access port may be in the form of a hole for permitting/allowing access to the surface for disposing the droplet of reaction mixture to the surface. A tip of a dispenser such as a droplet dispenser, e.g. pipette tip of a pipetting device, may be used to deliver the sample, magnetic particles, and/or one or more reaction reagents to the surface for disposing the droplet of reaction mixture. The sample, magnetic particles, and one or more reaction reagents may be sequentially delivered to the surface for disposing the droplet of reaction mixture. For example, a reaction reagent e.g. lysis buffer may be first dispensed, followed by the magnetic particles and then the sample. The sample, magnetic particles, and one or more reaction reagents may also be delivered via the first access port in a pre-mixed form where the sample, magnetic particles, and one or more reaction reagents are pre-mixed together prior to delivery via the first access port.

In various embodiments, the plurality of test units is positioned on the droplet manipulator such that the first access ports of the plurality of test units are spaced apart to be compatible for use with a conventional multichannel pipette. This may advantageously allow the sample, magnetic particles, and/or one or more reaction reagents to be concurrently dispensed onto the surface for disposing the droplets of reaction mixture.

In various embodiments, the test unit of the droplet manipulator further comprises a second access port configured to facilitate/allow delivery of a detection agent. The second access port may be in the form of a hole for permitting/allowing access to the surface for disposing the droplet of reaction mixture to the surface. A tip of a dispenser such as a droplet dispenser, e.g. pipette tip of a pipetting device, may be used to deliver the detection reagent to the surface for disposing the droplet of reaction mixture. The second access port may further be configured to facilitate delivery of one or more reagents e.g. a test substance. The test substance functions to react with a target substance in the sample and causes the detection reagent to produce a detectable signal, e.g. a visual signal. If the target substance is not present in the sample, no reaction with the test substance occurs and no detectable (e.g. visual) signal is produced by the detection reagent. The first access port and second access port may be arranged to be positioned at opposite ends of the test unit such that that the mixing element is disposed between the first and second access ports. The first access port and second access port advantageously facilitate delivery of various reagents to perform various kinds of assays using the system as a platform.

In various embodiments, the droplet manipulator is made of translucent or substantially transparent material to facilitate visualisation of the movement of the magnetic particles and the droplets through the droplet manipulator, and/or visualisation of the mixing of the reaction mixture, and/or visualisation of a visual signal is produced by the detection reagent.

In various embodiments, the system for performing an assay further comprises a guide member comprising at least one guide hole configured to be alignable with the first access port. The guide member may be configured to be detachably couplable to the droplet manipulator. The guide member may comprise a plurality of guide holes. At least one guide hole may be arranged to be aligned with a corresponding access port e.g. a first guide hole may be arranged to be aligned with a first access port of a test unit on the droplet manipulator and a second guide hole may be arranged to be aligned with a second access port of the test unit when the guide member is coupled to the droplet manipulator. Furthermore, in a sample loading mode, the guide hole may be aligned with a corresponding access hole of the droplet manipulator and a corresponding magnet on the base plate. One or more (or each) guide holes may be dimensioned such that a dispenser such as a droplet dispenser, e.g. a sample dispenser coupled thereto does not contact/touch the surface for disposing the droplet e.g. droplet of reaction mixture or droplet of detection reagent. That is, one or more (or each) guide holes may be dimensioned such that a tip of a dispenser such as a droplet dispenser, e.g. pipette tip of a pipetting device is allowed to deliver fluid e.g. fluid containing a sample, but is not allowed to contact the surface where the droplet e.g. droplet of reaction mixture is to be disposed. Advantageously, this may prevent the tip of the droplet dispenser from scratching and/or damaging the surface/substrate, which may impede movement of droplets over the surface.

In various embodiments, the test unit of the droplet manipulator further comprises a droplet holder configured to engage and hold the droplet of reaction mixture to facilitate observation. The droplet holder may comprise a contact surface e.g. hydrophilic contact surface for engaging and contacting a droplet. The contact surface of the droplet holder may be suitably profiled or shaped to effectively engage and contact the droplet. For example, the contact surface may have a concave profile for engaging and coupling to a convex surface of the droplet. Suitable profiles or shapes for the contact surface of the droplet holder may include but are not limited to a semi-circular shape, semi cylindrical shape, hemispherical shape, and the like.

In various embodiments, the droplet holder is further configured to facilitate removal of the magnetic particles from the droplet of reaction mixture. The droplet holder may be made from material which provides a hydrophilic surface such that the droplet holder is capable of anchoring the droplet for observation. The droplet holder may be chemically modified or treated to increase surface tension of the contact surface. For example, the droplet holder may be coated with polydopamine such that the contact surface of the droplet holder has a relatively higher surface tension for anchoring/keeping the droplet in position while magnetic particles are extracted from the droplet of reaction mixture. The droplet holder may be configured to move in tandem with the droplet manipulator along the alignment path. In various embodiments, as the droplet which is engaged to the droplet holder is moved away from the magnetic field applied by the magnet, the magnetic particles remain immobilised within the magnetic field of the magnet. As a result, the magnetic particles may be extracted from the droplet.

In various embodiments, the test unit of the droplet manipulator further comprises an observation window configured to facilitate observation of the droplet of reaction mixture. The observation window may allow visual observation of the droplet of reaction mixture when the droplet of reaction is positioned within the field of view of the observation window. For example, the observation may include observing whether a visual signal is produced by the detection reagent. The observation window may comprise a substantially transparent cover. The substantially transparent cover may have a convex shape to magnify the droplet of reaction mixture which may further facilitate observation. The droplet holder may be arranged to hold the droplet of reaction mixture under the observation window.

In various embodiments, the system for performing an assay is suitable for performing assays which produce visual signals e.g. colorimetric assays, chemiluminescent signals, electrochemical signals, or combinations thereof. The visual signals may be a visually observable colour signal (e.g. change in colour) or a fluorescent signal.

In one embodiment, the system is suitable for performing a Carba NP assay which detects presence of antibiotic-resistant bacteria e.g. Carbapenemase Producing Enterobacteriaceae (CPE) by measuring a change in pH when the antibiotic-resistant bacteria metabolize carbapenem, which is a class of highly effective antibiotic agents commonly used for the treatment of severe or high-risk bacterial infections. Antibiotic-resistant bacteria e.g. CPE produces carbapenemase enzyme which hydrolyzes an antibiotics agent e.g. carbapenum. If the bacteria are resistant to the antibiotic agent, the carbapenemase produced by the bacteria digests the antibiotic and reduces the pH of the reaction mixture/broth, causing the colour of the pH indicator, conventionally phenol red, to change from red to yellow. If the bacteria are susceptible to the antibiotic agent, the colour of the reaction mixture remains red.

In various embodiments, the reaction mixture comprises one or more of the following: a sample, one or more reaction reagents, and one or more detection reagents.

In various embodiments, the sample comprises a biological sample. The biological sample may be bacteria e.g. gram-negative or gram-positive bacteria. The bacteria may include, but is not limited to, Klebsiella pneumoniae, Escherichia coli, Acinetobacter baumanii, Bacillus cereus, Bacteroides fragilis, Citrobacter amalonaticus, Citrobacter freundii, Enterobacter aerogenes, Enterobacter cloacae, Klebsiella oxytoca, Morganella morganii, Proteus mirabilis, Proteus vulgaris, Providencia rettgeri, Providencia stuartii, Pseudomonas aeruginosa, Salmonella enterica, Serratia marcescens, Shigella flexneri, Stenotrophomonas maltophila and Ralstonia pickettii. The biological sample may be obtained from a subject such as clinical samples of urine and blood.

In various embodiments, the one or more reaction reagents may comprise a lysis reagent. For example, the lysis reagent may be a lysis buffer solution used for breaking open cells for use in molecular biology experiments that analyze the labile macromolecules of the cells.

In various embodiments, the one or more reaction reagent may comprise an activator to act as a cofactor. For example, a carbapenemase activator may be used to act as a cofactor to improve enzyme performance. The activator may be optional since some classes of carbapenemase exhibit hydrolytic activity without the need for a cofactor. If an activator is used, then the cofactor may be a divalent cation. The divalent cation may be a salt of manganese, cobalt, nickel, cadmium, mercury, zinc and mixtures thereof. In one embodiment, a zinc salt, such as zinc sulphate, is used as the activator. The carbapenemase activator may be used at a concentration of from about 0.01 mM to about 1 mM, from about 0.05 mM to about 0.9 mM, from about 0.1 mM to about 0.8 mM, from about 0.2 mM to about 0.7 mM, from about 0.3 mM to about 0.6 mM, or from about 0.4 mM to about 0.5 mM.

In one embodiment, the one or more reaction reagent may comprise an antimicrobial agent. The antimicrobial agent may be an antibiotic agent. The antibiotic agent may be carbapenem, which is a class of antibiotic agent commonly used for the treatment of severe or high-risk bacterial infection. Examples of carbapenem include but are not limited to imipenem, meropenem, ertapenem, doripenem, panipenem/betamipron, biapenem, tebipenem, razupenem, lenapenem, tomopenem, thienamycin.

In various embodiments, the detection reagent is capable of producing a visual signal indicating presence of a target substance. In one embodiment, the detection reagent is an indicator e.g. pH indicator. The detection reagent may be an indicator which include but is not limited to phenol red. The pH indicator may be any suitable indicator which shows a visible or recognisable colour transition when the pH changes from a first level to a second level. For example, the pH indicator may be a suitable indicator which shows a clear colour transition when the pH changes from about 4 to about 9, from about 4.5 to about 8.5, from about 5 to about 8, from about 5.5 to about 7.5, from about 6 to about 7, or from about 6.5 to about 7.

In various embodiments, the system for performing an assay is a magnetic digital microfluidics system/platform. A magnetic digital microfluidic system may advantageously provide a simple and easy way of manipulating small amounts of fluids for bioassays. A digital microfluidic platform is capable of manipulating droplets on a surface e.g. an open surface. Magnetic digital microfluidics are capable of utilising magnetic forces for manipulation of fluid in the form of droplets. This may provide a simple way of manipulating small amount of fluid for point-of-care diagnostic applications. Compared to conventional closed-channel microfluidic systems, a magnetic digital microfluidic platform eliminates bulky pumping and valving sytems, and hence is more well-suited for sample-to-answer point-of-care diagnostics.

In various embodiments, the system for performing an assay is portable. By portable, it is meant, among other things, that the system is capable of being transported relatively easily. The system may have an overall size and/or weight which allows it to be transported relatively easily.

In addition, the system may be suitable for use in both laboratory-based testing and on-site/out-field testing. The term “on-site” as used herein refers to performance of an activity at a site of particular concern. For example, the system may be used to perform the assay at a site/location where a sample material is obtained/located such that there is no need to transport the sample material back to the laboratory to be tested. It would be appreciated that during transportation of a sample to the laboratory for analysis, the sample may be subjected to changes such as contamination, degradation and the like. Therefore, the system may provide faster on-site analysis without undesirable changes to the integrity of the sample material.

In various embodiments, the system for performing an assay may be placed in a suitable environment for the assay to occur under optimal conditions of temperature, pressure, humidity, and the like. For example, the system may be placed in a pressure or humidity-controlled chamber e.g. incubator.

In various embodiments, the system for performing an assay may be operated manually or by other means that do not require an external power source. Advantageously, the system for performing an assay may be used for point-of-care applications in resource-limited environments where electricity is not readily available.

In various embodiments, the system for performing an assay may be compatible with automated control systems to provide a high throughput system for performing assays.

In various embodiments, a method of performing an assay is provided. The method of performing an assay may be performed using the system as disclosed herein. The method may comprise immobilising a droplet of reaction mixture doped with magnetic particles using a magnet disposed on a base member. The method may further comprise moving/sliding a droplet manipulator in relation to the base member along an alignment path. The droplet manipulator may comprise at least one test unit. Each test unit may comprise at least one mixing element. The method may further comprise inducing mixing of the droplet of reaction mixture immobilised by the magnet disposed on the base member using the mixing element in the test unit as the droplet manipulator is moved, e.g. slided back and forth, in relation to the base member along the alignment path. The method may further comprise observing changes to the reaction mixture.

In various embodiments, the method may comprise performing the assay simultaneously on a plurality of test units in the droplet manipulator. Each test unit in the plurality of test units may be configured to cooperate with a respective magnet disposed on the base member. This advantageously allows the assay to be performed simultaneously or concurrently on multiple samples.

In various embodiments, the method further comprises disposing the droplet of reaction mixture on a surface of the droplet manipulator. In various embodiments, the surface may be a surface that is coupled to the droplet manipulator. The surface of the droplet manipulator may be, or has been, chemically treated or modified to reduce surface energy. The reduction of surface energy may facilitate droplet formation on the surface and may facilitate movement of the droplet of reaction mixture along the surface.

In various embodiments, the method further comprises disposing the droplet of reaction mixture on the surface of the droplet manipulator via a first access port of the test unit. The step of disposing the droplet of reaction mixture may comprise disposing/delivering a sample, magnetic particles, and/or one or more reaction reagents via the first access port in the test unit. The step may comprise delivering the sample, magnetic particles, and one or more reaction reagents sequentially via the first access port. The step may also comprise delivering the sample, magnetic particles, and one or more reaction reagents in a pre-mixed form (i.e. premixed reaction mixture) where the sample, magnetic particles, and one or more reaction reagents are pre-mixed together prior to delivery via the first access port. The sample, magnetic particles, and/or one or more reaction reagents may be delivered using a dispensing device e.g. pipette to a single test unit or multichannel pipette to a plurality of test units. In various embodiments, a tip of the dispensing device is allowed to pass through the first access port in the test unit and deliver the sample, magnetic particles, and/or one or more reaction reagents to the surface of the droplet manipulator. In various embodiments, the sample and/or one or more reaction reagents that are deposited on the surface of the droplet manipulator form the droplet of reaction mixture.

In various embodiments, prior to delivering the sample, magnetic particles, and/or one or more reaction reagents via the first access port in the test unit, the method further comprises positioning the droplet manipulator relative to the base member such that the first access port on the droplet manipulator is substantially aligned to the magnet disposed on the base member. In various embodiments, such an alignment allows the sample, magnetic particles, and/or one or more reaction reagents that are delivered via the first access port to be positioned within the magnetic field applied by the magnet, e.g. above where the magnet is disposed on the base member.

In various embodiments, the base member comprises side walls which define the range of movement of the droplet manipulator along the alignment path on the base member, e.g. a first and second side walls disposed at opposite ends of the base member. To facilitate alignment, the first access port on the droplet manipulator may be arranged to be substantially aligned to the magnet disposed on the base member when the droplet manipulator is abutted against the first side wall of the base member. As such, the step of aligning the first access port on the droplet manipulator to the magnet disposed on the base member may comprise moving the droplet manipulator in relation to the base member in a first direction along the alignment path to abut the first side wall of the base member.

In various embodiments, where the sample, magnetic particles, and one or more reaction reagents are sequentially delivered via the first access port, the method may comprise first delivering the one or more reaction reagent, followed by the magnetic particles to prepare/prime the test unit. The one or more reaction reagent may comprise a lysis buffer, which is a buffer solution used for the purpose of breaking open cells for use in molecular biology experiments that analyze the labile macromolecules of the cells.

In various embodiments, the method further comprises disposing a droplet of detection reagent on the surface of the droplet manipulator via a second access port of the test unit. The step of disposing the droplet of detection reagent may comprise disposing/delivering a detection reagent via the second access port in the test unit. In various embodiments, the detection reagent that is delivered via the second access port is disposed at a different position on the surface of the droplet manipulator from the droplet of reaction mixture that is delivered via the first access port. The detection reagent may be delivered using a dispensing device e.g. pipette to a single test unit or multichannel pipette to a plurality of test units. A tip of the dispensing device is allowed to pass through the second access port in the test unit and deliver the detection reagents to the surface of the droplet manipulator. The method may further comprise delivering one or more reaction reagents via the second access port. The one or more reaction reagents to be delivered via the second access port may comprise an antimicrobial agent. The one or more reaction reagents to be delivered via the second access port may further comprise an activator compound for acting as a cofactor.

In various embodiments, prior to disposing the droplet of reaction mixture and droplet of detection reagent via the access ports e.g. prior to disposing/delivering the sample via the first access port in the test unit, the method comprises detachably coupling a guide member to the droplet manipulator. The guide member may comprise a plurality of guide holes. Each guide hole on the guide member is arranged to be aligned with an access port e.g. first access port of a test unit on the droplet manipulator when the guide member is coupled to the droplet manipulator. The method may comprise aligning a guide hole of the guide member to the access port through which the respective disposing step is to be carried out. The method may further comprise engaging a tip of a dispenser such as a droplet dispenser, e.g. pipette tip of a pipetting device, to the guide hole which is dimensioned such that the tip of the droplet dispenser does not contact the surface for disposing the droplet of reaction mixture when disposing the reaction mixture or detection reagent thereon. The method may further comprise delivering the sample via the first access port which is coupled to the guide hole. In various embodiments, the method further comprises detaching the guide member from the system prior to the step of moving the droplet manipulator in relation to the base member.

In various embodiments, the method further comprises interacting with the droplet of reaction mixture using the mixing element which comprises an array of pillars. The interaction between the droplet of reaction mixture and the mixing element may induce mixing of the sample and one or more reaction reagents within the droplet of reaction mixture. The step of interacting with the droplet of reaction mixture may comprise moving the droplet manipulator in relation to the base member along the alignment path such that the mixing element moves toward and interacts with the droplet of reaction mixture immobilised by the magnet disposed on the base member.

For example, the step may involve moving the droplet manipulator in relation to the base member in a second direction opposite to the first direction along the alignment path. In various embodiments, this results in the mixing element moving in tandem with the droplet manipulator in the second direction towards the droplet of reaction mixture. It will be appreciated that in various embodiments, as the droplet manipulator is moving in relation to (i.e. relative to) the base member in the second direction along the alignment path, the immobilised droplet of reaction mixture is moving in relation to (i.e. relative to) the surface of the droplet manipulator along the first direction along the alignment path. The step of interacting with the droplet of reaction mixture may comprise moving the droplet manipulator back and forth in the first and second direction in relation to the base member along the alignment path such that the mixing element is moved back and forth in the first and second direction in relation to the droplet of reaction mixture immobilised by the magnet disposed on the base member, thereby enhancing mixing of the droplet of reaction mixture.

In various embodiments, the method further comprises incubating the droplet of reaction mixture. In various embodiments, incubation allows the sample and the one or more reaction reagents in the droplet of reaction mixture to react. For example, the sample may comprise a bacterial sample and the one or more reaction reagent may comprise a lysis buffer. Thus, in various embodiments, incubation may allow the bacterial cells to be fully lysed by the lysis buffer. Incubating the droplet of reaction mixture may comprise allowing the droplet of reaction mixture to sit on the surface of the droplet manipulator for a period of time which may range from about 30 minutes to about 3 hours. Incubating the droplet of reaction mixture may further comprise placing the droplet of reaction mixture in a suitable environment to allow the reaction in the reaction mixture to proceed under suitable conditions e.g. temperature, pressure, humidity level, and the like. For example, placing the droplet of reaction mixture in a pressure and/or humidity-controlled environment may prevent evaporation of the droplet.

In various embodiments, the method further comprises producing a visual signal by the detection reagent upon detecting presence of a target substance. The step of producing a visual signal by the detection reagent may comprise adding the detection reagent and/or one or more reaction reagent to the droplet of reaction mixture. The detection reagent and/or one or more reaction reagent may be disposed as a droplet on the surface of the droplet manipulator under the second access port. The step of adding the detection reagent and/or one or more reaction reagent to the droplet of reaction mixture may comprise merging the droplet of detection reagent and/or one or more reaction reagent with the droplet of reaction mixture to form a merged droplet. The step of adding the detection reagent and/or one or more reaction reagent to the droplet of reaction mixture may comprise moving the droplet manipulator in relation to the base member along the alignment path to merge the droplet of reaction mixture with the droplet of detection reagent. For example, the droplet manipulator may move in relation to the base member along the alignment path such that the droplet of detection reagent moves toward and merges with the droplet of reaction mixture which is immobilised by the magnet disposed on the base member.

For example, the step of adding the detection reagent and/or one or more reaction reagent to the droplet of reaction mixture may involve moving the droplet manipulator in relation to the base member in the second direction along the alignment path. In various embodiments, this results in the droplet of detection reagent moving in tandem with the droplet manipulator in the second direction towards the droplet of reaction mixture. It will be appreciated that in various embodiments, as the droplet manipulator is moving in relation to (i.e. relative to) the base member in the second direction along the alignment path, the immobilised droplet of reaction mixture is moving in relation to (i.e. relative to) the surface of the droplet manipulator along the first opposite direction along the alignment path.

In various embodiments, the method further comprises mixing the merged droplet, i.e. droplet of reaction mixture after adding the detection reagent and/or one or more reaction reagent, which comprises interacting with the merged droplet using the mixing element. The interaction between the merged droplet and the mixing element induces mixing of the sample, detection reagent and one or more reaction reagents within the merged droplet. The step of interacting with the merged droplet may comprise moving the droplet manipulator in relation to the base member along the alignment path such that the mixing element moves toward and interacts with the merged droplet immobilised by the magnet disposed on the base member, thereby inducing further mixing.

For example, the step may comprise moving the droplet manipulator in relation to the base member in the first direction along the alignment path. This results in the mixing element moving in tandem with the droplet manipulator in the first direction towards the droplet of reaction mixture. It will be appreciated that in various embodiments, as the droplet manipulator is moving in relation to (i.e. relative to) the base member in the first direction along the alignment path, the immobilised droplet of reaction mixture is moving in relation to (i.e. relative to) the surface of the droplet manipulator along the second opposite direction along the alignment path. The step of interacting with the droplet of reaction mixture may comprise moving the droplet manipulator back and forth in the first and second direction in relation to the base member along the alignment path such that the mixing element is moved back and forth in the first and second direction in relation to the droplet of reaction mixture immobilised by the magnet disposed on the base member, thereby enhancing mixing of the droplet of reaction mixture.

In various embodiments, the step of moving the droplet manipulator in relation to the base member along the alignment path comprises moving the droplet manipulator in relation to the base member which is in a stationary position. In various embodiments, the magnet disposed on the base member may be fixed in position on the base member, i.e. not moveable with respect to the base member. During performance of the assay, the droplet doped with magnetic particles may be immobilised in a fixed position relative to the magnet and does not substantially displace from the fixed position. That is, the droplet doped with magnetic particles may be immobilised in the fixed position relative to the magnet until the assay is completed. Once the assay is completed, the droplet may be displaced from the fixed position, e.g. by transferring to another position to facilitate observation of the assay result.

In various embodiments, the method further comprises engaging and holding the droplet of reaction mixture using a droplet holder to facilitate observation. The test unit of the droplet manipulator comprises a droplet holder configured to engage and hold the droplet of reaction mixture to facilitate observation. The droplet holder may comprise a contact surface for engaging and holding a droplet. In various embodiments, the droplet holder has been chemically treated to provide a hydrophilic contact surface for engaging and holding the droplet of reaction mixture. In various embodiments, the step may comprise moving the droplet manipulator in relation to the base member along the alignment path to engage and hold the droplet of reaction mixture using the droplet holder.

In various embodiments, to facilitate engagement and contact of the droplet holder with the droplet of reaction mixture, the droplet holder may be arranged to engage and contact the droplet of reaction mixture when the droplet manipulator is abutted against the second side wall of the base member. As such, the step of engaging and holding the droplet of reaction mixture using a droplet holder may comprise moving the droplet manipulator in relation to the base member in the second direction along the alignment path to abut the second side wall of the base member. This results in the droplet holder moving in tandem with the droplet manipulator in the second direction towards the droplet of reaction mixture. It will be appreciated that in various embodiments, as the droplet manipulator is moving in relation to (i.e. relative to) the base member in the second direction along the alignment path, the immobilised droplet of reaction mixture is moving in relation to (i.e. relative to) the surface of the droplet manipulator along the first opposite direction along the alignment path.

In various embodiments, the method further comprises removing the magnetic particles from the droplet of reaction mixture via movement of the droplet manipulator along the alignment path. In various embodiments, the magnetic particles are removed from the droplet of reaction mixture prior to observation of the assay results. Removal of the magnetic particles may facilitate observation or visualization of the assay results in the droplet of reaction mixture. In various embodiments, the step of removing the magnetic particles from the droplet of reaction mixture comprises moving the droplet manipulator in relation to the base member along the alignment path, thereby moving the droplet of reaction mixture away from its immobilised position within the magnetic field of the magnet.

In one example embodiment, the step of removing the magnetic particles from the droplet of reaction mixture may comprise moving the droplet manipulator in relation to the base member in the first direction along the alignment path away from the second side wall of the base member. In various embodiments, this results in the droplet of reaction mixture which is engaged to the droplet holder moving in tandem with the droplet manipulator in the first direction along the alignment path. In various embodiments, the magnetic particles which are immobilised within the magnetic field of the magnet are removed as the droplet of reaction mixture which is engaged to the droplet holder is moving in relation to (i.e. relative to) the base member in the first direction away from the magnetic field of the magnet.

In various embodiments, prior to observation of the assay results in the droplet of reaction mixture, the method further comprises incubating the droplet of reaction mixture. In various embodiments, incubation allows the sample and the one or more reaction reagents in the droplet of reaction mixture to react. Incubating the droplet of reaction mixture may comprise allowing the droplet of reaction mixture to sit on the surface of the droplet manipulator for a period of time which may range from about 30 minutes to about 3 hours. Incubating the droplet of reaction mixture may further comprise placing the droplet of reaction mixture in a suitable environment to allow the reaction in the reaction mixture to proceed under suitable conditions e.g. temperature, pressure and/or humidity-controlled environment, and the like. For example, placing the droplet of reaction mixture in a humidity-controlled environment may prevent evaporation of the droplet.

In various embodiments, the method further comprises observing via an observation window in the test unit, the droplet of reaction mixture that is engaged and held by the droplet manipulator. In one embodiment, the droplet holder may be positioned in relation to the observation window, e.g. below the observation window such that the visual signal e.g. colour change in the droplet of reaction mixture is observable from the observation window.

### BRIEF DESCRIPTION OF FIGURES

FIG. 1A is a schematic perspective view drawing of a system/device for performing an assay in an example embodiment.

FIG. 1B is a photograph of a base member in the example embodiment.

FIG. 1C is a schematic bottom view drawing of a droplet manipulator in the example embodiment.

FIG. 1D is a photograph of the droplet manipulator in the example embodiment.

FIG. 2A is a schematic bottom view drawing of a droplet manipulator in an example embodiment.

FIG. 2B is a magnified view of a test unit in the example embodiment.

FIG. 2C is a schematic perspective view drawing of a guiding piece in an example embodiment.

FIG. 3A is a schematic perspective view drawing showing sample loading into a system for performing an assay in an example embodiment.

FIG. 3B is a schematic perspective view drawing showing movement direction of a droplet manipulator relative to a base member in the example embodiment.

FIG. 3C is a schematic perspective view drawing showing movement direction of droplets relative to the droplet manipulator when the droplet manipulator moves in the direction as shown in FIG. 3B.

FIG. 3D is a first schematic side view drawing of the system showing movement of the droplet manipulator and relative movement of the droplets in the example embodiment.

FIG. 3E is a second schematic side view drawing of the system showing movement of the droplet manipulator and relative movement of the droplets in the example embodiment.

FIG. 3F is a third schematic side view drawing of the system showing movement of the droplet manipulator and relative movement of the droplets in the example embodiment.

FIG. 4A is a schematic drawing of a system for performing an assay in an example embodiment.

FIG. 4B is a schematic drawing of the system showing sample loading in the example embodiment.

FIG. 4C is a schematic drawing of the system showing movement of a droplet manipulator in relation to a base member in the example embodiment.

FIG. 4D is a schematic drawing of the system showing mixing of a droplet of reaction mixture by the droplet manipulator in the example embodiment.

FIG. 4E is a schematic drawing of the system showing merging of the droplet of reaction mixture with a droplet of detection reagent in the example embodiment.

FIG. 4F is a schematic drawing of the system showing movement of the droplet manipulator in relation to the base member in the example embodiment.

FIG. 4G is a schematic drawing of the system showing mixing of a merged droplet by the droplet manipulator in the example embodiment.

FIG. 4H is a schematic drawing of the system showing coupling of the merged droplets by the droplet holders in the example embodiment.

FIG. 4I is a schematic drawing of the system showing removal of magnetic particles from the merged droplets in the example embodiment.

FIG. 5A is a photograph of a platform for performing a Carba NP assay.

FIG. 5B is a photograph of the platform showing mixing of samples by moving droplets back and forth under a mixer provided in each detection unit.

FIG. 5C is a photograph of the platform showing sample droplets merged with reagent droplets.

FIG. 5D is a photograph of the platform showing merged droplets being mixed under the mixer.

FIG. 5E is a photograph of the platform showing the merged droplets moved to respective observation windows.

FIG. 5F is a photograph of the platform showing magnetic particles extracted/moved out from the merged droplets.

FIG. 6A is a photograph of a digital microfluidic point-of-care platform for performing a Carba NP assay with prepared reagents and magnetic particles placed on the platform.

FIG. 6B is a photograph of the platform with a 3D printed top plate cover placed on top of the platform.

FIG. 6C is a photograph of the platform with reaction droplet and detection droplet merged.

FIG. 6D is a photograph of the platform showing colour changes in the droplets after 30 minutes.

FIG. 6E is a photograph of the platform showing colour changes in the droplets after 1 hour.

FIG. 6F is a photograph of the platform showing color changes in the droplets after 2 hours.

FIG. 6G is a photograph of the platform showing the final results of a Carba NP assay with a test group and a control group labelled.

FIG. 7 is a photograph of a droplet manipulator showing 12 test units with reaction droplets for different samples.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural, and optical changes may be made without deviating from the scope of the invention. Example embodiments are not necessarily mutually exclusive as some may be combined with one or more embodiments to form new exemplary embodiments.

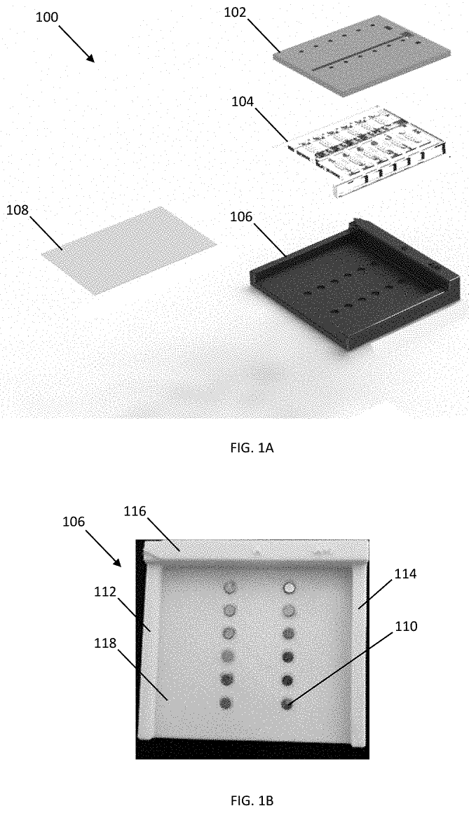

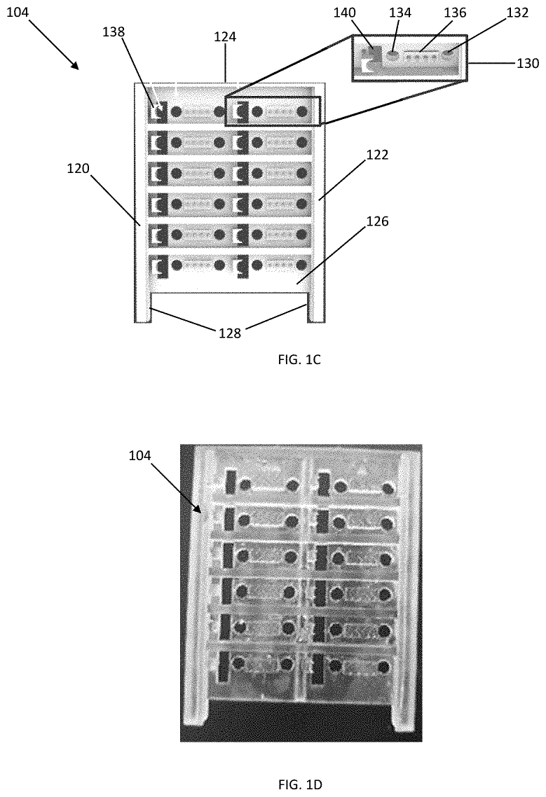

FIG. 1A is a schematic perspective view drawing of a system/device 100 for performing an assay in an example embodiment. The system 100 for performing an assay is a multiplexed CPE detection system comprising a magnetic digital microfluidic system that is capable of conducting multiple Carba NP assays in parallel in droplets. The system 100 comprises four main components, a guide member/guiding piece 102, a droplet manipulator 104, a base member/base plate 106 and a sheet member 108 for disposing one or more droplets of reaction mixture e.g. glass coverslip. The guiding piece 102 and the droplet manipulator 104 can be coupled/combined into a single component. The guiding piece 102 is used to guide loading of a sample during an assay preparation phase, and the other three components 104, 106 and 108 are used to manipulate droplets during the assay.

FIG. 1B is a photograph of the base member 106 in the example embodiment. The base plate 106 contains an array of magnets e.g. 110 to manipulate droplets using magnetic digital microfluidics. The base plate 106 comprises a first side wall 112, a second side wall 114, and a third side wall 116 surrounding a flat bed 118. The first side wall 112 and the second side wall 114 are disposed at opposite sides of the flat bed 118 and are connected or joined by the third side wall 116. The three side walls 112, 114, 116 restrict movement of the droplet manipulator 104 so that the droplet manipulator does not overshoot, i.e. does not move outside of the base member 106. Magnets e.g. 110 are embedded into holes provided on the flat bed 118. The magnets e.g. 110 interact with magnetic particles added to droplets e.g. droplets of reaction mixture that sit on the coverslip e.g. glass coverslip 108. Together with the droplet manipulator 104, the magnets e.g. 110 control the movement and operation of droplets. The glass coverslip 108 is coated with substances (e.g. Teflon AF) that renders down the surface energy of the coverslip 108. As a result, the droplets could move easily on the surface of the coverslip 108.

FIG. 1C is a schematic bottom view drawing of the droplet manipulator 104 in the example embodiment. FIG. 1D is a photograph of the droplet manipulator 104 in the example embodiment. The droplet manipulator 104 comprises micro physical structures and chemically modified features to facilitate droplet manipulation. The bottom aspect of the droplet manipulator 104 contains small structures and surface modifications to facilitate droplet manipulation. The droplet manipulator 104 is made of translucent materials so that a user can visualize movement of the magnetic particles and the droplets through the droplet manipulator 104.

The droplet manipulator 104 comprises a first side wall 120, a second side wall 122, and a third side wall 124 surrounding a base 126. The first side wall 120 and the second side wall 122 are disposed at opposite sides of the base 126 and are connected or joined by the third side wall 124. The three sidewalls 120, 122, 124 comprise slots 128 e.g narrow slots which function as holders for the coverslip 108. The coverslip 108 is arranged to be slided into the slots 126 from an opening side of the droplet manipulator 104.

The droplet manipulator 104 further comprises a plurality of test units/testing units e.g. 130. A magnified view of one testing unit 130 is also shown in FIG. 1C. The base 126 of the droplet manipulator 104 is divided into two columns of repeating testing units e.g. 130. Each testing unit 130 comprises a first access port/hole 132 and a second access port/hole 134 for delivering fluid e.g. liquid. Reagents such as lysis buffer and magnetic particles are dispensed on top of the coverslip 108 through one access hole e.g. first access hole 132, and detection reagent is dispensed through the other access hole e.g. second access hole 134. Each testing unit 130 further comprises a mixing element e.g. an array of pillars 136 for droplet mixing disposed between the two access holes 132, 134. As the droplet moves through the array of pillars 136, the droplet is stretched and retracted repetitively, which induces mixing inside the droplet. At the end of each testing unit 130, a droplet holder e.g. semi-circular droplet holder 138 is disposed under an observation window e.g. rectangular observation window 140. The entire bottom surface of the droplet manipulator 104 is coated with Teflon. In addition, the semi-circular droplet holder 138 is coated with polydopamine which renders its surface hydrophilic so that it is capable of anchoring the droplet for observation.

The device 100 is designed for parallel multiplexed analysis of CPE using a droplet based Carba NP assay, capable of analyzing 6 clinical samples (or 12 reactions) concurrently with a simple fluidic operation. All the 12 reactions can be performed at the same time by simply sliding the droplet manipulator 104 from right to left on top of the base plate 106.

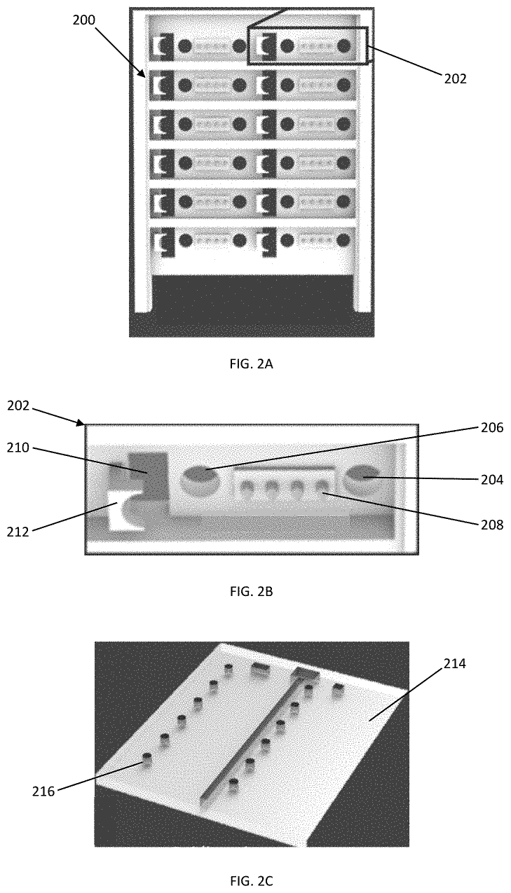

FIG. 2A is a schematic bottom view drawing of a droplet manipulator 200 in an example embodiment. The droplet manipulator 200 comprises 12 units of testing units e.g. 202. FIG. 2B is a magnified view of the test unit 202 in the example embodiment. The magnified view of the test unit 202 provides a closeup of different structures for droplet holding, droplet mixing, and droplet adding. In each testing unit 202, a first access port 204 and a second access port 206 are provided to allow reagents and samples to be added to an underlying surface e.g. glass coverslip. Between the two access ports 204, 206, a mixing element 208 e.g. an array of pillars 208 is provided as mixers to passively mix the fluids by stretching and slinging the droplet. An observation window 210 is provided adjacent to the second access port 206 at the end of the testing unit 202 through which an operator/user could observe the color of droplets. A droplet holder 212 e.g. a semi-circular droplet holder 212 is coated with polydopamine to enhance surface tension (or to provide a surface with relatively high surface tension) to keep the droplet engaged to the droplet holder while the magnetic particles are extracted from the droplet. The glass coverslip was coated with 1% Teflon AF solution by a spin coating method and is arranged to be placed between the droplet manipulator 200 and a base member/platform (compare 106 of FIG. 1A). The base member is embedded with an array of 12 magnets, and the locations of the magnets correspond to the location of the droplets to be disposed on the glass coverslip. FIG. 2C is a schematic perspective view drawing of a guiding piece 214 in an example embodiment. The guiding piece 214 is arranged to be put on top of the droplet manipulator during sample loading, i.e. bacteria transferring process. The guiding piece 214 contains 12 holes e.g. 216 with a pre-determined size/diameter. The locations of the 12 holes e.g. 216 in the guiding piece 214 correspond to the locations of the first access ports e.g. 204 on the droplet manipulator 200, i.e. the initial location where the lysis buffer droplet is disposed on the coverslip. The size of the holes e.g. 216 in the guiding piece 214 restricts how deep a tip of a pipette could be inserted into, ensuring the bacterial colony at the tip of the pipette is fully submerged in the droplet but does not contact the coating on the glass coverslip to prevent scratching of the coating.

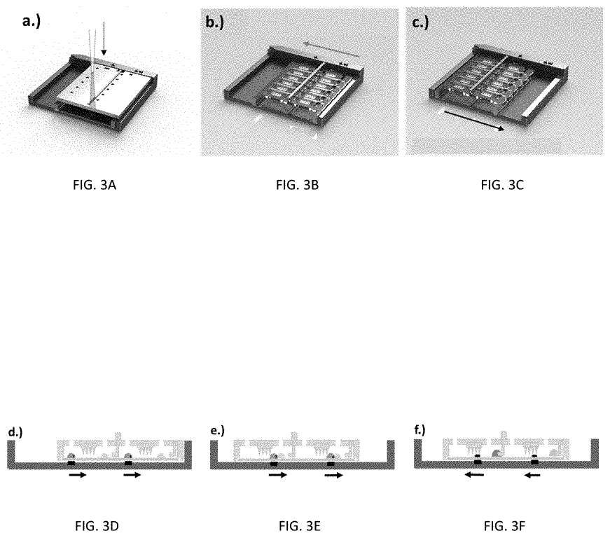

An assay e.g. Carba NP may be performed on the magnetic digital microfluidic platform as disclosed herein in three main steps. FIG. 3A TO FIG. 3C outline the three main steps of parallel multiplexed analysis of CPE (Carbapenemase Producing Enterobacteriaceae) using a droplet-based Carba NP assay.

FIG. 3A is a schematic perspective view drawing showing sample loading into a system for performing an assay in an example embodiment. FIG. 3B is a schematic perspective view drawing showing movement direction of a droplet manipulator relative to a base member in the example embodiment. FIG. 3C is a schematic perspective view drawing showing movement direction of droplets relative to the droplet manipulator when the droplet manipulator moves in the direction as shown in FIG. 3B.

FIG. 3D is a first schematic side view drawing of the system showing movement of the droplet manipulator and relative movement of the droplets in the example embodiment. FIG. 3E is a second schematic side view drawing of the system showing movement of the droplet manipulator and relative movement of the droplets in the example embodiment. FIG. 3F is a third schematic side view drawing of the system showing movement of the droplet manipulator and relative movement of the droplets in the example embodiment.

The Carba NP may be performed on the magnetic digital microfluidic platform in three major steps as follows:

First, all required reagents are dispensed through the access ports on the droplet manipulator (see FIG. 3A). Two columns of reactions are prepared on the device, with testing reactions in the left column and the control reactions on the right. In each reaction, one lysis buffer droplet and one solution A droplet are added. In the testing reaction, carbapenem (e.g. imipenem) is added to the solution A droplet. Bacterial isolates are added to the lysis buffer droplets using pipette tips. The pipette tip is used to pick the bacterial colony, and the guiding piece ensures the pipette is inserted to a designated depth at the right location.

Second, the droplet manipulator, which holds the glass coverslip to move together/in tandem with the droplet manipulator, is used to move the lysis buffer droplet to merge with the solution A droplet (see FIGS. 3B, 3D and 3E). During the process, the magnetic particles added to the lysis buffer droplet are pulled by the magnet array which in turn pulls the droplet into motion. Once merged with the solution A droplet, the combined droplet is moved back and forth below the mixer teeth to passively mix the components within. After mixing, the combined droplet is incubated for up to 2 hours.

Third, the combined droplet is moved to the observation window where there is a polydopamine-coated droplet holder to keep the droplet in position while the magnetic particles are extracted from the droplet (see FIGS. 3C and 3F). All droplets are manipulated at the same time in parallel, all reactions are performed concurrently, and all results are observed in one go from the observation windows in the end.

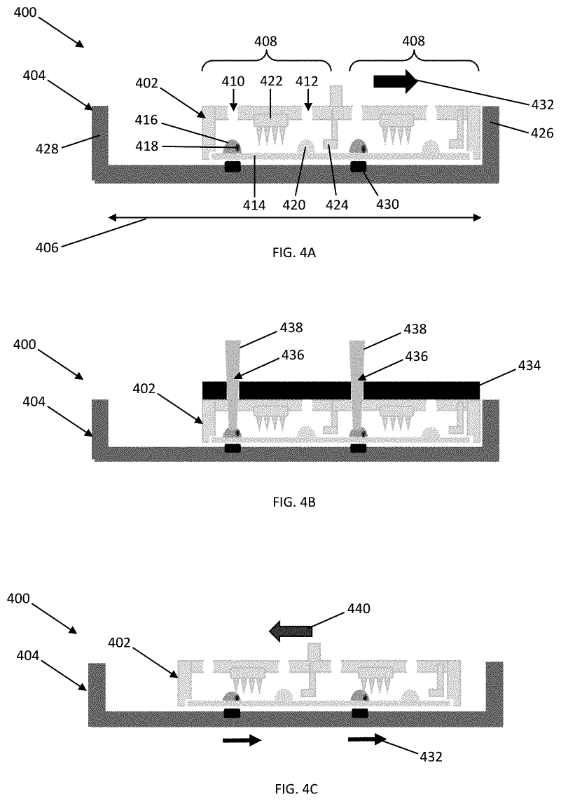

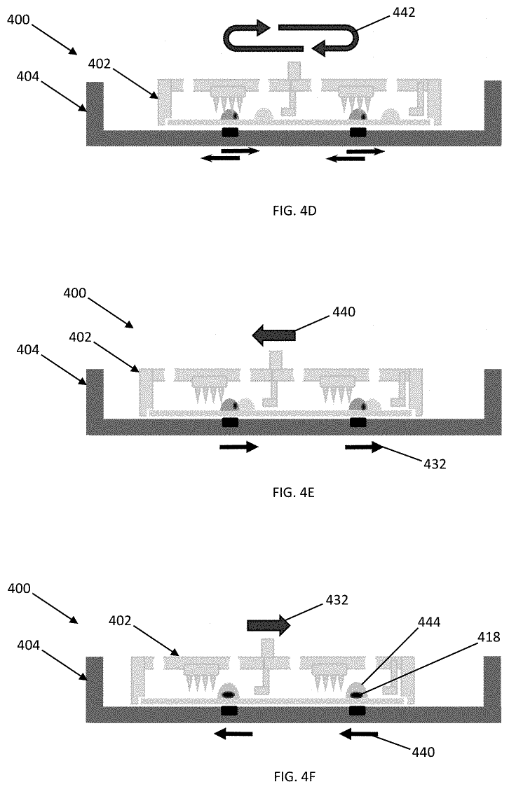

FIG. 4A is a schematic drawing of a system 400 for performing an assay in an example embodiment. The system 400 comprises a droplet manipulator 402 slidably mounted on a base member 404. The droplet manipulator 402 is arranged to slide in relation to the base member 404 along an alignment path 406.

The droplet manipulator 402 comprises a plurality of test units e.g. 408, each test unit 408 comprising a first access port 410 and a second access port 412 for facilitating delivery of fluid onto a surface of a sheet member 414 for disposing droplets. The first access port 410 is arranged to facilitate delivery of a sample, magnetic particles and/or one or more reaction reagents to form a droplet of reaction mixture 416 doped with magnetic particles 418. The second access port 412 is arranged to facilitate delivery of a detection reagent and/or one or more reaction reagents to form a droplet of detection reagent 420. The droplet manipulator 402 further comprises a mixing element 422 positioned between the first access port 410 and the second access port 412. The mixing element 422 comprises a plurality of pillars e.g. overhanging pillars extending from an underside/undersurface of the droplet manipulator 402. The mixing element 422 is arranged to interact with the droplets e.g. 416, 420 disposed on the surface of the sheet member 414 to induce mixing of the droplets e.g. 416, 420. The droplet manipulator 402 further comprises a droplet holder 424 disposed at one end of the test unit 408 adjacent or near to the second access port 412. The droplet holder 424 is configured to engage and maintain/hold a droplet in position under an observation window (not shown) to facilitate observation.

The base member 404 comprises a first side wall 426 and a second side wall 428 defined at opposite sides of the base member 404. The first side wall 426 and the second side wall 428 define the range of movement of the droplet manipulator 402 along the alignment path 406. The base member 404 further comprises a plurality of magnets e.g. 430 embedded on the base member 404. Each magnet 430 is arranged to immobilise the droplet of reaction mixture 416 doped with magnetic particles 418 within a magnetic field applied by the magnet 430. In the example embodiment, the magnet 430 is arranged to immobilise the droplet of reaction mixture 416 doped with magnetic particles 418 at a position directly above the magnet 430.

During performance of an assay, the droplet manipulator 402 is placed on top of the base member 404 and is pushed/moved in a first direction 432 to contact/abut against the first side wall 426 (i.e. right wall) of the base member 404. The sheet member e.g. a glass coverslip 414 is slid in between the droplet manipulator 402 and the base member 404 to provide the surface for disposing the droplets. The magnet 430 embedded in the base plate 404 is configured to align with the first access port 410 (i.e. left access hole) of each test unit 408 when the droplet manipulator 402 is abutted against the first side wall 426. The droplet manipulator 402 may comprise six pairs of test units e.g. 408 for parallel testing of multiple samples. The six pairs of test units e.g. 408 may be arranged into two columns on the droplet manipulator 402. The spacing between each test unit 408 is configured to match the spacing between adjacent pipettes in a multiple channel micropipette. All reagents in one column of test units e.g. 408 could be dispensed in together using the multiple channel micropipette. One or more reaction reagents e.g. lysis buffer is dispensed onto the surface of the sheet member 414 e.g. Teflon-coated coverslip through the first access port 410 (i.e. left access hole). The detection reagent is dispensed onto the surface of the sheet member 414 e.g. Teflon-coated coverslip through the second access port 412 (i.e. right access hole). The magnetic particles 418 are dispensed into the droplet of reaction mixture 416 comprising the lysis buffer. All liquids spontaneously form droplets once dispensed onto the surface of the sheet member 414 due to the relatively low surface energy of the surface e.g. Teflon-coated coverslip.

FIG. 4B is a schematic drawing of the system 400 showing sample loading in the example embodiment. A guide member 434 e.g. guiding piece comprising a plurality of guide holes/guiding holes e.g. 436 is placed/removably coupled on top of the droplet manipulator 402. The guiding holes e.g. 436 are arranged to align with the first access port 410 (i.e. left access hole) when the guide member 434 is coupled to the droplet manipulator 402. A sampler 438 (e.g. a loop or pipette tip) with bacterial isolates from clinical specimens is inserted into the droplet of reaction mixture 416 containing the lysis buffer droplet through the guiding hole 436. The size of the guiding hole 436 is configured such that the tip of the sampler 438 is submerged in the droplet of reaction mixture 416 but does not touch the surface of the sheet member 414 (i.e. surface of the glass coverslip). The size of the guiding hole 436 may be adjusted according to the size of the sampler 438.

FIG. 4C is a schematic drawing of the system 400 showing movement of the droplet manipulator 402 in relation to the base member 404 in the example embodiment. In the example embodiment, the droplet manipulator 402 is moved in a second direction 440 along the alignment path 406 (i.e. towards the left). The mixing elements e.g. 422 are also moved in tandem with the droplet manipulator 402 in the second direction 440 toward the droplets of reaction mixture e.g. 416. The second direction 440 is opposite to the first direction 432 along the alignment path 406. As the droplet manipulator 402 is moving in the second direction 440, the magnets e.g. 430 immobilise/hold the droplet of reaction mixture 416 (i.e. sample droplet) in position above the magnets e.g. 430. As a result, the droplet of reaction mixture 416 moves along the surface of the sheet member 414 in the first direction 432 to the right towards the droplet manipulator in a relative motion. The direction of motion of the droplet of reaction mixture 416 relative to the surface of the sheet member 414 is depicted as arrows below the base member 404.

FIG. 4D is a schematic drawing of the system 400 showing mixing of the droplet of reaction mixture 416 by the droplet manipulator 402 in the example embodiment. The droplet manipulator 402 is moved back and forth in the first direction 432 and second direction 440 so that the sample droplets 416 repetitively pass through the pillar array of the mixing element 422 for enhanced mixing. The back and forth motion of the droplet manipulator 402 is depicted in FIG. 4D as arrows 442. After mixing, the sample droplets 416 are incubated to allow the bacterial cells to be fully lysed.

FIG. 4E is a schematic drawing of the system 400 showing merging of the droplet of reaction mixture 416 with the droplet of detection reagent 420 in the example embodiment. After incubation, the droplet manipulator 402 is moved in the second direction 440 along the alignment path 406 (i.e. to the left). The droplet of detection reagent 420 are also moved in tandem with the droplet manipulator 402 in the second direction 440 toward the droplets of reaction mixture e.g. 416. As the droplet manipulator 402 is moving in the second direction 440, the magnets e.g. 430 immobilise/hold the droplet of reaction mixture 416 (i.e. sample droplet) in position above the magnets e.g. 430. As a result, the sample droplets 416 travel in the first direction 432 (i.e. to the right) in a relative motion to merge with the droplet of detection reagent 420 to form merged droplets e.g. 444.

FIG. 4F is a schematic drawing of the system 400 showing movement of the droplet manipulator 402 in relation to the base member 404 in the example embodiment. After droplet merging, the droplet manipulator 402 is moved in the first direction 432 along the alignment path 406 (i.e. to the right). The mixing elements e.g. 422 are also moved in tandem with the droplet manipulator 402 in the first direction 432 toward the merged droplets e.g. 444. As the droplet manipulator 402 is moving in the first direction 432, the magnets e.g. 430 immobilise/hold the merged droplets e.g. 444 in position above the magnets e.g. 430. As a result, the merged droplets e.g. 444 travel in the second direction 440 towards the array of pillars of the mixing element 422 (i.e. to the left) in a relative motion.

FIG. 4G is a schematic drawing of the system 400 showing mixing of the merged droplet 444 by the droplet manipulator 402 in the example embodiment. The droplet manipulator 402 is moved back and forth in the first direction 432 and second direction 440 so that the merged droplets e.g. 444 travel left and right under the array of pillars of the mixing element 422 to facilitate mixing.

FIG. 4H is a schematic drawing of the system 400 showing coupling of the merged droplets e.g. 444 by the droplet holders e.g. 424 in the example embodiment. After mixing, the droplet manipulator 402 is pushed/moved in the second direction 440 towards, or to contact/abut against the second side wall 428 (i.e. left wall) of the base member 404. As the droplet manipulator 402 is moving in the second direction 440 towards the second side wall 428, the droplet holders e.g. 424 engage and contact the merged droplets e.g. 444.

FIG. 4I is a schematic drawing of the system 400 showing removal of the magnetic particles e.g. 418 from the merged droplets in the example embodiment. The droplet manipulator 402 is moved in the first direction 432 (i.e. to the right) along the alignment path 406. The merged droplets e.g. 444 which are engaged to the droplet holders e.g. 424 are also moved in tandem with the droplet manipulator 402 in the first direction 432. As a result, the magnetic particles e.g. 418 travel in the second direction 440 (i.e. to the left) in relative motion. As the droplet holders 424 are holding on to the merged droplets e.g. 444, the magnetic particles e.g. 418 are split from the merged droplets e.g. 444 as the droplet manipulator 402 is moved in the first direction 432. The removal of magnetic particles e.g. 418 from the merged droplets e.g. 444 facilitates visualization of the merged droplets e.g. 444. The merged droplets e.g. 444 that are engaged and held by the droplet holders e.g. 424 are positioned directly under the observation window. Prior to observation, the merged droplets e.g. 444 are incubated to allow detection reactions to take place in humidity-controlled environment for a period of time from about 30 min to about 3 hours. The results of the assay may be observed via the observation window e.g. change in color of the detection reagent.

The system 400 is a magnetic digital microfluidic platform which is suitable for performing various assays which produce results in the form of visual signals. In a Carba NP assay performed on the system 400, the system 400 is first primed with reagents. 10 μL of lysis buffer and 3.5 μL magnetic particles e.g. 418 are dispensed onto the glass coverslip 414 through the left access holes e.g. 410 of both test units in each row of test units e.g. 408, and the droplets form droplets on their own. 10 μL of the detection reagent droplet containing phenol red and 0.1 mM ZnSO₄ are dispensed onto the glass coverslip 414 through the right access holes e.g. 412 of both test units in each row of test units e.g. 408, and the droplets form droplets on their own. The detection reagent droplet in the left test unit contains 6 mg/mL Imipenem antibiotics, whereas the one in the right test unit does not, thereby serving as a control. All of the solutions were prepared before starting the experiment. The results of the assay are shown via the colour of the merged droplets e.g. 444 after incubation. For a sample with bacterial strain that is resistant to the antibiotic tested, a color change is observed in the merged droplet e.g. red to yellow for a phenol red detection reagent. In FIG. 4I, the merged droplet e.g. 444 in the left test unit changed colour as depicted by a change in shade of grey relative to FIG. 4H, while the merged droplet e.g. 444 in the right test unit remained the same colour as depicted by the same shade of grey relative to FIG. 4H. For the assay to be valid, the color of the merged droplet e.g. 444 in the right test unit serving as the control should remain the same colour.

### EXAMPLE

The droplet manipulations required to perform a Carba NP assay on the system/platform have been demonstrated in the various example embodiments.

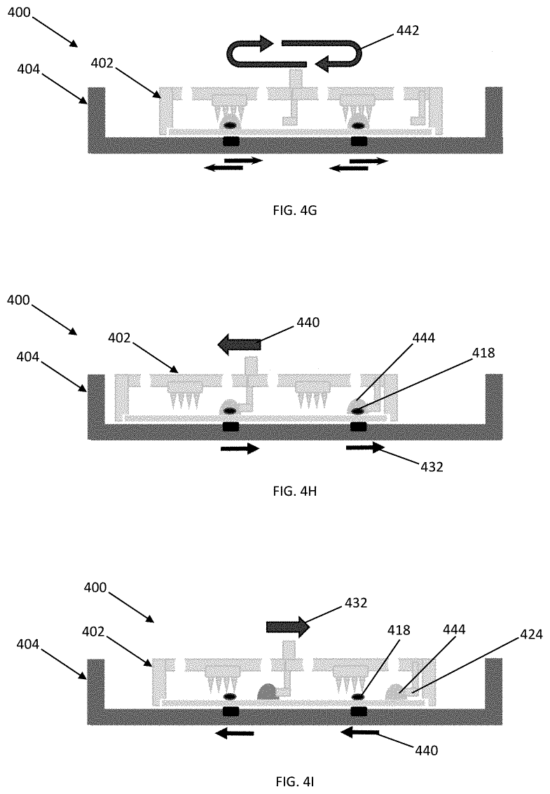

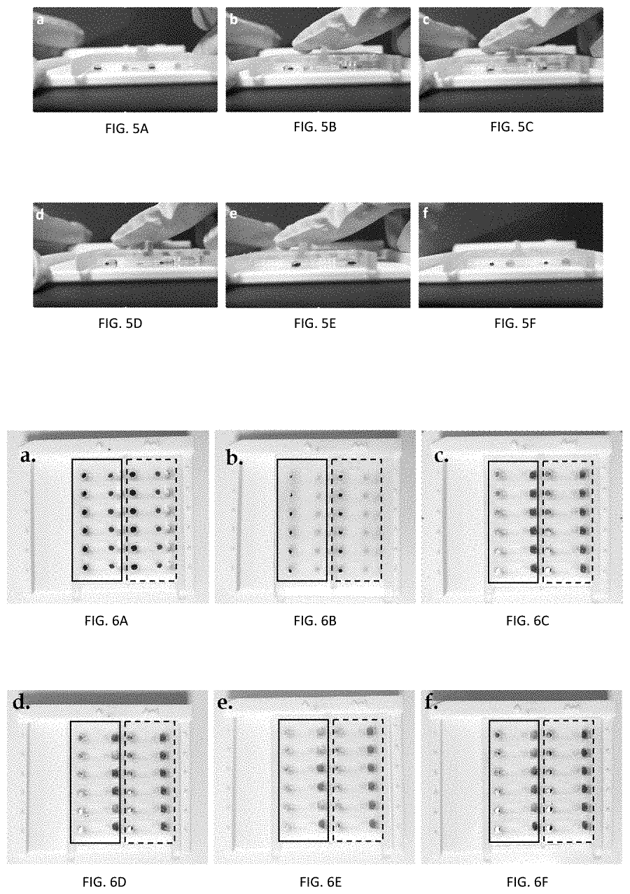

FIG. 5A is a photograph of a platform for performing a Carba NP assay. As shown in FIG. 5A, samples, reagents and magnetic particles were dispensed onto respective test units/detection units of the platform to form droplets. Lysis buffer and bacteria sample were added into each detection unit at a first position (see left hand side position of each detection unit in FIG. 5A). Detection reagent (i.e. Solution A with Imipenem for the test group or Solution A without Imipenem for the control group) was added to a second position (see right hand side position of each detection unit in FIG. 5A). After magnetic particles were added into the first position, Carba NP droplet manipulation was performed as shown in the subsequent FIGS. 5B to 5F. FIG. 5B is a photograph of the platform showing mixing of the samples by moving the droplets back and forth under a mixer provided in each detection unit. FIG. 5C is a photograph of the platform showing the sample droplets merged with the reagent droplets. FIG. 5D is a photograph of the platform showing the merged droplets being mixed under the mixer. FIG. 5E is a photograph of the platform showing the merged droplets moved to respective observation windows. FIG. 5F is a photograph of the platform showing magnetic particles extracted/moved out from the merged droplets.

The device/platform was first primed with reagents. 10 μL of lysis buffer and 3.5 μL of magnetic particles were dispensed onto the glass coverslip through the left access holes of both detection units in each row, and the droplets formed droplets on their own. 10 μL of the detection reagent droplet containing phenol red and 0.1 mM ZnSO₄ were dispensed onto the glass coverslip through the right access holes of both detection units in each row, and the droplets formed droplets on their own. The detection reagent droplet in the left detection unit contained 6 mg/mL Imipenem antibiotics, whereas the one in the right detection unit did not contain Imipenem antibiotics. All of the solutions were prepared before starting the experiment.

The multiplexed Carba NP assays were carried out. Twenty-four droplets containing reagents and magnetic particles were filled in the holes by using multichannel pipette. Five CPE samples (1^{st}-5^{th}) and one negative sample (6^{th}), counting from top to bottom respectively, were picked and immersed in lysis buffer droplets by using the 3D printed top plate cover to hold the sample tips. After a simple slide of the platform, the yellow color change was observed (Table. 1 and FIG. 6A-FIG. 6G). Table 1 shows a list of samples of bacteria isolates from the top to bottom row and the color changes shown after the 2 hours incubation on the Digital Microfluidic Point-of-Care Platform.

TABLE 1
List of samples of bateria isolates
and results of the Carba NP assay

| | | Sol A + | |
|---|---|---|---|
| No. | Samples | Imi | Sol A |
| 1 | Escherichia coli NCTC (IMP-type) | Yellow | Red |
| 2 | Escherichia coli 6013499989 (blaKPC+) | Yellow | Red |
| 3 | Klebsiella pneumonia 2073318014 (blaNDM+) | Orange | Red |
| 4 | Klebsiella pneumonia ATT BAA-1705 (KPC+) | Orange | Red |
| 5 | Escherichia coli MBRL 235 (NDM+) | Yellow | Red |
| 6[—] | Escherichia coli- C3 (6123-57039) | Red | Red |

FIG. 6A to FIG. 6G are a series of photographs taken at various time points of performing a Carba NP assay using the platform as disclosed herein.

FIG. 6A is a photograph of a digital microfluidic point-of-care platform for performing a Carba NP assay with the prepared reagents and magnetic particles placed on the platform. As shown in FIG. 6A, the samples in the test group were loaded into the detection units on the left side of the platform (as demarcated by the rectangle in solid line) while the samples in the control group were loaded into the detection units on the right side of the platform (as demarcated by the rectangle in dotted line). 10 μL of lysis buffer and 3.5 μL of magnetic particles were dispensed onto the glass coverslip through the left access holes of each detection units to form droplets (see 1^{st} and 3^{rd} columns of the platform in FIG. 6A). 10 μL of the detection reagent droplet containing phenol red and 0.1 mM ZnSO₄ were dispensed onto the glass coverslip through the right access holes of each detection unit to form red colour droplets (see 2^{nd} and 4^{th} columns of the platform in FIG. 6A).

FIG. 6B is a photograph of the platform with a 3D printed top plate cover placed on top of the platform. The top plate cover or guide member comprising 12 guide holes was detachably coupled to the platform such that each of the 12 guide holes were aligned to the left access hole of each detection unit. The guide holes are dimensioned such that a tip of a dispenser such as a droplet dispenser, e.g. pipette tip of a pipetting device is allowed to deliver fluid e.g. fluid containing a sample, but is not allowed to contact the surface where the droplet e.g. droplet of reaction mixture is to be disposed. This may prevent the tip of the droplet dispenser from scratching and/or damaging the surface/substrate, which may impede movement of droplets over the surface. Five CPE samples (1^{st}-5^{th} rows of the platform) and one negative sample (6^{th} row of the platform), counting from top to bottom respectively, were picked and immersed in the lysis buffer droplets by using the 3D printed top plate cover to hold the sample tips.

FIG. 6C is a photograph of the platform with reaction droplet and detection droplet merged. The magnetic particles are extracted out of the merged droplet after moving the platform left and right. As shown in FIG. 6C, the extracted magnetic particles were positioned on the 1^{st} and 3^{rd} columns of the platform while the merged droplets were positioned on the 2^{nd} and 4^{th} columns of the platform. As the photograph of FIG. 6C was taken immediately after the reaction droplets and detection droplets were merged, the merged droplets still display the red colour of the phenol red indicator.

FIG. 6D is a photograph of the platform showing color changes in the droplets after 30 minutes. The test group sample in the 2^{nd} row of the platform changed colour from red to yellow and the test group sample in the 5^{th} row of the platform changed colour from red to orange after 30 minutes of merging the reaction droplets and detection droplets. The rest of the droplets remained red in colour after 30 minutes of merging the reaction droplet and detection droplet.

FIG. 6E is a photograph of the platform showing color changes in the droplets after 1 hour. The test group sample in the 2^{nd} row of the platform remained yellow and the test group sample in the 5^{th} row of the platform changed colour from orange to yellow after 1 hour of merging the reaction droplets and detection droplets. In addition, the test group sample in the 1^{st} row of the platform changed colour from red to yellow after 1 hour of merging the reaction droplets and detection droplets. The rest of the droplet remained red in colour after 1 hour of merging the reaction droplets and detection droplets.

FIG. 6F is a photograph of the platform showing color changes in the droplets after 2 hours. The test group samples in the 1^{st}, 2^{nd} and 5^{th} rows of the platform remained yellow after 2 hours of merging the reaction droplets and detection droplets. In addition, the test group samples in the 3^{rd} and 4^{th} rows of the platform changed colour from red to orange after 2 hours of merging the reaction droplets and detection droplets. The test group sample in the 6^{th} row remained red in colour after 2 hours of merging the reaction droplet and detection droplet.

FIG. 6G is a photograph of the platform showing the final results of the Carba NP assay with the test group and control group labelled. The test units on the left column belong to the test group which has an antibiotic agent (Imipenem antibiotics) added. The test units on the right column belong to the control group without addition of the antibiotic agent. The bacterial strains of the samples and the final colours of the respective merged droplets after 2 hours of merging the reaction droplets and detection droplets are summarised in the above Table 1. For the assay to be valid, the color of the droplets in the control group should remain red (see 4^{th} column of the platform).

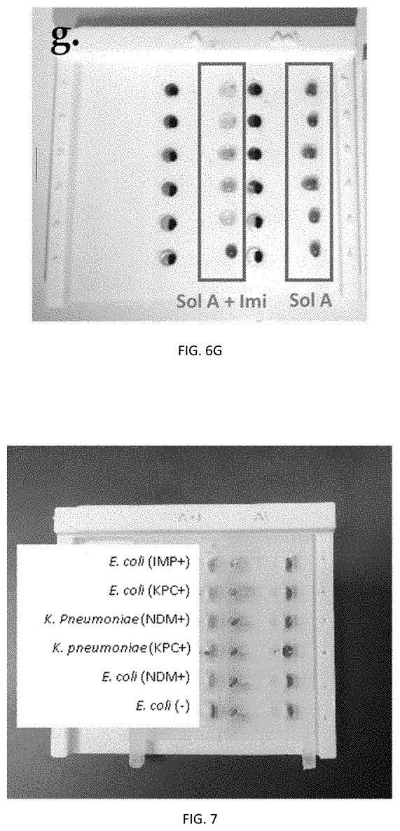

FIG. 7 is a photograph of a droplet manipulator showing 12 test units with reaction droplets for different samples. The results of the assay were based on color changes in accordance to different bacterial strains. The genotypes of the bacterial strains were characterized using molecular techniques. The first five strains, which comprised of two species with 3 CRE subgroups (IMP, KPC and NDM) are CRE₊ (CRE positive) (CRE=carbapenem-resistant enterobacteriaceae, IMP=Imipenem-resistant Pseudomonas, KPC=Klebsiella Pneumoniae Carbapenemase, NDM=New Delhi Metallo-beta-lactamase). The last strain was a CRE-(CRE negative) E. Coli (Escherichia coli) strain. Each strain was tested in a pair of testing units in one row. The left unit contained antibiotics for testing. The right unit was antibiotic-free and served as a control. In the left testing units, the color of the top 5 droplets changed from red to yellow, indicating resistance to carbapenems. The color of the last droplet remained red, suggesting that the strain was susceptible to carbapenems. For the assay to be valid, the color of the control droplets in the right column should remain red.

### Applications

Embodiments of the disclosure provided herein provide a system and method of performing an assay. In various embodiments, an assay may be performed by adding samples and reagents required for the assay into test units provided in a droplet manipulator.

Advantageously, embodiments of the disclosed system and method provide flexible fluidic control as the reaction mixture is performed on a surface disposed on the droplet manipulator. Accordingly, in various embodiments, the samples and reagents do not have to follow pre-defined fluid flow paths such as those found in conventional channel-based microfluidic platforms. Embodiments of the disclosed system and method also provide flexibility in the types of assays which can be performed. Reagents for various assays can be easily introduced onto the surface of the droplet manipulator via the access ports provided in each testing unit of the droplet manipulator.

Even more advantageously, embodiments of the disclosed system and method may be capable of simplifying assay workflow and reduces the assay time by testing multiple samples (i.e. multiplexed detection) on a magnetic digital microfluidic platform with just a single action. As the reaction of the assay may be performed in the microdroplet form, consumption of reagents is significantly reduced. For example, the magnetic digital microfluidic system may be capable of conducting multiple assays e.g. Carba NP assays in parallel in droplets. This may overcome the problems of conventional detection assays such as the Carba NP assay, which are tedious to perform and typically only analyze one clinical isolate at a time, which is time-consuming in diagnostic settings where a large number of clinical isolates need to be tested. The magnetic digital microfluidic platform may be applied in multiplexed diagnostics of infections caused by carbapenemase resistance in gram-negative bacilli.

Even more advantageously, embodiments of the disclosed system and method can be operated manually or by other means that do not require an external power source, which is an important consideration for point-of-care applications in resource-limited environments where electricity is not readily available. Embodiments of the disclosed system and method may also be compatible with automated control systems to provide a high throughput system for performing assays.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A magnetic digital microfluidic system for performing an assay, the system comprising,
a base member comprising at least one magnet disposed thereon; the magnet being configured to immobilise a droplet of reaction mixture doped with magnetic particles; and
a droplet manipulator configured to be moveably mountable on the base member, said droplet manipulator comprising at least one test unit, each test unit comprising at least one mixing element for mixing the droplet of reaction mixture;
wherein the mixing element is arranged to induce mixing as the droplet manipulator is moved in relation to the base member along an alignment path.

2. The system according to claim 1, further comprising a surface for allowing the droplet of reaction mixture to be disposed thereon.

3. The system according to claim 2, wherein the surface is a detachable surface and the droplet manipulator is configured to detachably couple with the detachable surface.

4. The system according to claim 2, wherein the test unit comprises,
a first access port for allowing delivery of a sample, magnetic particles, and/or one or more reaction reagents to said surface.

5. The system according to claim 1, wherein the mixing element comprises an array of pillars being arranged to interact with the droplet of reaction mixture to induce mixing.

6. The system according to claim 2, wherein the test unit further comprises a second access port for allowing delivery of a detection reagent to said surface.

7. The system according to claim 1, wherein the test unit further comprises a droplet holder configured to engage and hold the droplet of reaction mixture to facilitate observation.

8. The system according to claim 7, wherein the droplet holder comprises a hydrophilic contact surface for engaging and holding the droplet of reaction mixture.

9. The system according to claim 7, wherein the droplet holder is further configured to facilitate removal of the magnetic particles from the droplet of reaction mixture via movement of the droplet manipulator along the alignment path.

10. The system according to claim 1, wherein the test unit further comprises an observation window configured to facilitate observation of the droplet of reaction mixture.

11. The system according to claim 1, wherein the droplet manipulator comprises a plurality of test units for performing the assay on a plurality of samples simultaneously, each test unit being configured to cooperate with a respective magnet disposed on the base member.

12. The system according to claim 4, further comprising a guide member configured to be detachably couplable to the droplet manipulator, said guide member comprising at least one guide hole configured to be alignable with the first access port,
wherein the guide hole is dimensioned such that a droplet dispenser inserted thereto does not contact the surface where the droplet is to be disposed.

13. (canceled)

14. A method of performing an assay with the magnetic digital microfluidic system of claim 1, the method comprising,
moving the droplet manipulator in relation to the base member along an alignment path to induce mixing of a droplet of reaction mixture immobilised by the magnet disposed on the base member, wherein the reaction mixture is doped with magnetic particles; and
observing changes to the reaction mixture.

15. The method according to claim 14, further comprising, prior to the moving step,
disposing a droplet of reaction mixture on a surface that is coupled to the droplet manipulator via a first access port of the test unit.

16. The method according to claim 15, further comprising, subsequent to said moving step,
disposing a droplet of detection reagent on said surface via a second access port of the test unit;
further moving the droplet manipulator in relation to the base member along an alignment path to merge the droplet of reaction mixture with the droplet of detection reagent; and
optionally further moving the droplet manipulator in relation to the base member along an alignment path to further mix the merged droplet.

17. The method according to claim 15, further comprising, prior to the each of said disposing step(s),
detachably coupling a guide member to the droplet manipulator; and
aligning a guide hole of the guide member to the access port through which the respective disposing step is to be carried out,
wherein the guide hole is dimensioned such that a droplet dispenser does not contact the surface of the droplet manipulator when disposing the reaction mixture or detection agent thereon.

18. The method according to claim 17, further comprising, detaching the guide member from the system prior to each of said moving step(s).

19. The method according to claim 14, further comprising,
moving the droplet manipulator in relation to the base member along an alignment path to engage and hold the droplet of reaction mixture with a droplet holder;
moving the droplet manipulator in relation to the base member along an alignment path to remove the magnetic particles from the droplet of reaction mixture; and
observing via an observation window in the test unit, the droplet of reaction mixture that is engaged and held by the droplet manipulator.

20. The method according to claim 14, further comprising performing the assay on a plurality of samples simultaneously on a plurality of test units in the droplet manipulator, wherein each test unit is being configured to cooperate with a respective magnet disposed on the base member.
